(19) United States
(12) Patent Application Publication
(10) Pub. No.: US 2026/0242832 A1
(43) Pub. Date: Aug. 20, 2026

(54) BIOELECTRIC HYBRID SYSTEM PRODUCING LACTIC ACID FROM CARBON DIOXIDE

(71) Applicant: KOREA ADVANCED INSTITUTE OF SCIENCE AND TECHNOLOGY, Daejeon (KR)

(72) Inventors: Hyunjoo LEE, Daejeon (KR); Sang Yup LEE, Daejeon (KR); ChangHun HWANG, Daejeon (KR); Jaewon LEE, Daejeon (KR); Jeageon LEE, Daejeon (KR)

(73) Assignee: KOREA ADVANCED INSTITUTE OF SCIENCE AND TECHNOLOGY, Daejeon (KR)

(21) Appl. No.: 19/448,224

(22) Filed: Jan. 14, 2026

(30) Foreign Application Priority Data
Feb. 14, 2025 (KR) 10-2025-0019285

(51) Int. Cl. C12P 7/56; C12N 9/00; C12N 9/02; C12N 9/04; C12N 9/06; C12N 9/78; C12N 15/52; C12N 15/70; C25B 3/07; C25B 3/26; C25B 15/08

(52) U.S. Cl. CPC C12P 7/56; C12N 9/0006; C12N 9/0028; C12N 9/0093; C12N 9/78; C12N 9/93; C12N 15/52; C12N 15/70; C12Y 101/01027; C12Y 105/01015; C12Y 305/04009; C12Y 603/04003; C25B 3/07; C25B 3/26; C25B 15/081; C12N 2800/101

(57) ABSTRACT

Disclosed is a bioelectric hybrid system for producing lactic acid from carbon dioxide. The recombinant microorganism can produce lactic acid using, as carbon sources, carbon dioxide and formic acid electrochemically produced from carbon dioxide, and thus is useful for converting various high value-added compounds from carbon dioxide.

### CROSS REFERENCE TO RELATED APPLICATIONS

This application claims priority to Korean Patent Application No. 10-2025-0019285 filed Feb. 14, 2025, of which the entire content is incorporated by reference herein.

### SEQUENCE LISTING

The content of the electronically submitted sequence listing, file name: Q317274_SEQLIST-ST26.xml; size: 17,041 bytes; and date of creation: Jan. 14, 2026, filed herewith, is incorporated herein by reference in its entirety.

### BACKGROUND OF THE INVENTION

### Field of the Invention

The present invention relates to a bioelectric hybrid system for producing lactic acid from carbon dioxide, and more specifically, to a bioelectric hybrid system for producing lactic acid from carbon dioxide only based on combination of electrode system for electrochemically producing formic acid from carbon dioxide with a lactic acid fermentation system using recombinant microorganisms that synthesize lactic acid from carbon dioxide and formic acid.

### Description of the Related Art

The increase in carbon dioxide emissions resulting from the use of fossil fuels is emerging as a major cause of climate change. To address this issue, technologies that convert carbon dioxide into beneficial compounds are essential for building sustainable industries. In particular, electrochemical carbon dioxide reduction (ECO₂R) has attracted attention due to high conversion efficiency and scalability thereof, but is limited to producing primarily simple C₁ and C₂ compounds. Improving selectivity to produce compounds with more complicated structures poses technical challenges. Furthermore, biological carbon dioxide conversion is capable of producing a variety of compounds in mild environments, but has limited efficiency due to issues such as low carbon dioxide solubility and additional energy consumption.

Bioelectric hybrid systems are being proposed as an innovative alternative for carbon dioxide conversion. These systems provide a flexible platform for producing a variety of compounds based on combination of electrochemical approaches with microorganisms. In particular, electrochemically produced formic acid (FA) is attracting attention for the high production efficiency and high water solubility thereof. Formic acid may be supplied to microbial systems and converted into compounds having more complicated structures. In a previous study, the present inventors demonstrated that a bioelectric hybrid system using Cupriavidus necator directly produced polyhydroxybutyrate from carbon dioxide (Korean Patent No. 2,000,755). Furthermore, the present inventors constructed a recombinant microorganism capable of growing solely in the presence of carbon dioxide and formic acid by introducing and enhancing the metabolic pathway that synthesizes pyruvate from carbon dioxide and formic acid (Korean Patent No. 2,2690858).

Meanwhile, lactic acid is a key precursor to biodegradable polylactic acid (PLA) and is an important industrial compound used in eco-friendly plastics and various organic synthesis applications. Producing lactic acid solely from carbon dioxide could unlock new possibilities for converting carbon dioxide into high-value chemicals, providing a significant breakthrough in sustainable and environmentally friendly carbon dioxide conversion and carbon-based compound production.

Accordingly, as a result of great efforts to develop the first bioelectric hybrid system capable of directly producing lactic acid by conversion of carbon dioxide, the present inventors have found that, when a CO₂ electrolyzer that produces formic acid from carbon dioxide is combined with a recombinant microorganism that has been metabolically improved to convert formic acid and carbon dioxide into lactic acid, lactic acid can be efficiently produced using only carbon dioxide as a raw material. Based on this finding, the present invention has been completed.

### SUMMARY OF THE INVENTION

Therefore, the present invention has been made in view of the above problems, and it is one object of the present invention to provide a recombinant microorganism capable of producing lactic acid from carbon dioxide and formic acid.

It is another object of the present invention to provide a method of producing lactic acid from formic acid and carbon dioxide using the recombinant microorganism.

It is another object of the present invention to provide a bioelectric hybrid system device capable of producing lactic acid solely from carbon dioxide by combination of the recombinant microorganism with an electrode system that electrochemically produces formic acid from carbon dioxide.

In accordance with one aspect of the present invention, the above and other objects can be accomplished by the provision of a recombinant microorganism capable of producing lactic acid from carbon dioxide and formic acid, wherein a gene encoding L-lactate dehydrogenase is introduced into or amplified in a host microorganism having a central carbon assimilation pathway.

In accordance with another aspect of the present invention, provided is a method of producing a beneficial substance, including (a) culturing the recombinant microorganism using formic acid and carbon dioxide as a carbon source to produce a beneficial substance, and (b) recovering the produced beneficial substance.

In accordance with another aspect of the present invention, provided is a method of producing lactic acid from carbon dioxide using a bioelectric hybrid system, the method including: (a) converting carbon dioxide to formic acid through an electrochemical reduction reaction; and (b) fermenting a microorganism capable of biosynthesizing lactic acid from formic acid and carbon dioxide using the formic acid converted in step (a), to produce lactic acid.

In accordance with another aspect of the present invention, provided is a bioelectric hybrid system device for producing lactic acid from carbon dioxide, including (i) an electrolysis reactor for converting carbon dioxide into formic acid by an electrochemical reduction reaction, and (ii) a biosynthesis reactor fluidly connected to the electrolysis reactor and producing lactic acid from formic acid through fermentation of a microorganism in an electrode solution containing formic acid.

### Advantageous Effects

According to the present invention, the recombinant microorganism can produce lactic acid using, as carbon sources, carbon dioxide and formic acid electrochemically produced from carbon dioxide, and thus is useful for converting carbon dioxide into various high value-added compounds.

### BRIEF DESCRIPTION OF THE DRAWINGS

The above and other objects, features and other advantages of the present invention will be more clearly understood from the following detailed description taken in conjunction with the accompanying drawings, in which:
(a) of FIG. 1 is a schematic diagram illustrating a core metabolic pathway for producing lactic acid using carbon dioxide and formic acid (FA) of a recombinant microorganism and the genes, enzymes, and metabolites involved in the metabolic pathway, (b) of FIG. 1 shows cell growth and lactic acid (LA) production measured when the recombinant microorganism is cultured in a flask using carbon dioxide, formic acid (FA), and glucose (Glc) as carbon sources, and (c) of FIG. 1 shows the cell growth and lactic acid (LA) production measured when the recombinant microorganism is cultured in a fermenter using only formic acid and carbon dioxide as carbon sources;

FIG. 2 is a schematic diagram illustrating the plasmid p518FA1_Strepto_ldhA, constructed by inserting genes involved in carbon dioxide and formic acid assimilation, a formate dehydrogenase derived from the genus Candida, a mutant of a formate dehydrogenase derived from the genus Arabidopsis, and lactate dehydrogenase genes derived from the genus Streptococcus;
(a), (b) and (c) of FIG. 3 show the cell growth when the recombinant microorganism FC15 (a) and the parent strain AK01 (b) were cultured at high cell densities in the presence of only carbon dioxide and formic acid, and the ¹³C-labeled lactate ratio in the pre-culture and fermenter culture (c) when ¹³C-labeled glucose was pre-cultured and only formic acid and carbon dioxide were provided as carbon sources in the fermenter;
(a) of FIG. 4 is a schematic diagram illustrating a bioelectric hybrid system according to the present invention, and (b) of FIG. 4 shows the formic acid production under various current density conditions in a physiologically compatible negative electrode solution;

FIG. 5 shows the lactic acid production using only carbon dioxide in a bioelectric hybrid system according to the present invention under various initial inoculation cell densities, wherein (a) and (b) have an initial inoculation cell density of 1.5, (c) and (d) have an initial inoculation cell density of 3.3, and (e) and (f) have an initial inoculation cell density of 5.0; and

FIG. 6 shows the lactic acid production using only carbon dioxide in a bioelectric hybrid system under various current density conditions, wherein (a) and (b) have a current density condition of −90 mA cm⁻², (c) and (d) have a current density condition of −105 mA cm⁻², (e) and (f) have a current density condition of −120 mA cm⁻², and (g) and (h) have a current density condition of −140 mA cm⁻².

### DETAILED DESCRIPTION OF THE INVENTION

Unless defined otherwise, all technical and scientific terms used herein have the same meanings as appreciated by those skilled in the field to which the present invention pertains. In general, the nomenclature used herein is well-known in the art and is ordinarily used.

Formic acid (FA) is a C1 compound that is capable of highly efficiently producing carbon dioxide by electrochemical conversion. Formic acid is present as a liquid at room temperature and thus is easily stored and transported. Furthermore, formic acid may be used as a reducing agent for carbon source assimilation of cells via formate dehydrogenase (Fdh). Due to these advantages, formic acid has attracted attention as a carbon source for microbial metabolism. In a previous study, the present inventors designed and validated a novel metabolic pathway for converting formic acid, a C1 compound, into a beneficial multi-carbon compound (Korean Patent No. 2,000,755). Furthermore, the present inventors constructed a recombinant microorganism capable of growing solely on carbon dioxide and formic acid by introducing and enhancing a metabolic pathway for synthesizing pyruvate from carbon dioxide and formic acid (Korean Patent No. 2,690,858).

In the present invention, a recombinant microorganism capable of growing solely in the presence of carbon dioxide and formic acid (Korean Patent No. 2,690,858) was further metabolically engineered. The present inventors also improved a culture medium and culture method to be combined with an electrode system for producing formic acid, and found that the improved recombinant Escherichia coli could produce lactic acid solely from carbon dioxide.

In the recombinant microorganism constructed in Korean Patent No. 2,690,858, formic acid and carbon dioxide are introduced in the form of pyruvate into the central metabolic pathway. To produce lactic acid from formic acid and carbon dioxide based on the recombinant microorganism (parent strain), lactate dehydrogenase, which converts pyruvate to lactate, was introduced. Furthermore, endogenous lactate dehydrogenase genes (e.g., ldhA, dld, and lldD in E. coli) may be induced in low pH environments and under anaerobic conditions and may also convert lactic acid produced in the reverse reaction to pyruvate. For this reason, these genes were removed. In addition, the production of lactic acid from pyruvate was enhanced by introducing a gene encoding 1-lactate dehydrogenase from the genus Streptococcus.

Accordingly, in one aspect, the present invention is directed to a recombinant microorganism capable of producing lactic acid from carbon dioxide and formic acid, wherein a gene encoding L-lactate dehydrogenase is introduced into or amplified in a host microorganism having a central carbon assimilation pathway.

The recombinant microorganism may be characterized by a deletion of the gene encoding the endogenous lactate dehydrogenase.

In the present invention, the L-lactate dehydrogenase may be an enzyme capable of converting pyruvate to lactic acid.

As used herein, the term “central carbon assimilation pathway” refers to a metabolic pathway involved in the assimilation of central carbons, such as formic acid and/or carbon dioxide.
(a), (b) and (c) of FIG. 1 are schematic diagram illustrating the key metabolic pathways involved in the assimilation of carbon dioxide and formic acid in a recombinant microorganism, along with the genes, enzymes, and metabolites involved therein. In the present invention, the formic acid assimilation pathway is a cycle in which microorganisms assimilate carbon dioxide and formic acid into pyruvate containing three carbon atoms. (a), (b) and (c) of FIG. 1 illustrate the genes, coenzymes, and energy transfer substances involved in the formic acid assimilation pathway of Escherichia coli.

In one embodiment of the present invention, a recombinant microorganism was constructed by deleting the endogenous lactate dehydrogenase genes from a host microorganism having a formic acid and carbon dioxide assimilation pathway and introducing a gene encoding a heterologous 1-lactate dehydrogenase capable of converting pyruvate to lactate from the host microorganism.

In the present invention, the formic acid assimilation pathway can synthesize carbon compounds of C3 or higher through combination with a central carbon assimilation pathway. The host microorganism may have i) an endogenous central carbon assimilation pathway or ii) an exogenous central carbon assimilation pathway.

The gene encoding 1-lactate dehydrogenase involved in the lactic acid production may be a gene encoding 1-lactate dehydrogenase from the genus Streptococcus, which may be involved in the production of lactic acid from pyruvate, and may include a base sequence represented by SEQ ID NO: 6.

In the present invention, the recombinant microorganism may be characterized by a deletion of genes (ldhA, dld, lldD) encoding the lactate dehydrogenase of the host cell.

The genes (ldhA, dld, lldD) encoding the lactate dehydrogenase of the host cell may be ldhA, dld, or lldD genes. lldD was removed to prevent the redistribution and reuptake of l-LA, and the dld gene, which exhibits d-LA productivity, was removed to increase the selectivity of l-LA.

The recombinant microorganism of the present invention may further include a gene encoding at least one selected from the group consisting of formate-tetrahydrofolate ligase, methenyl tetrahydrofolate cyclohydrolase, methylene-tetrahydrofolate dehydrogenase, formate dehydrogenase, and a formate dehydrogenase mutant.

The gene encoding formate-tetrahydrofolate ligase may include the sequence of SEQ ID NO: 1, the gene encoding methenyltetrahydrophorate cyclohydrolase may include the sequence of SEQ ID NO: 2, the methylene-tetrahydrophorate dehydrogenase may include the sequence of SEQ ID NO: 3, and the formate dehydrogenase may include the sequence of SEQ ID NO: 4. Alternatively, the formate dehydrogenase mutant may include the sequence of SEQ ID NO: 5 (Table 1).

TABLE 1

| | | Origin |
|---|---|---|
| Target gene | NCBI information | microorganism |
| ftfL | Formate-tetrahydrofolate ligase, CP001298 REGION: | Methylobacterium |
| (SEQ ID NO: | 434499-436172 [SEQ ID NO: 1] | extorquens CM4 |
| 1) | 5′- | |
| | atgccctcagatatcgagatcgcccgcgcggcgaccctgaagccgatcgccca | |
| | ggtcgccgaaaagctcggcatcccggacgaggcgcttcacaattacggcaagc | |
| | acatcgccaagatcgaccacgacttcatcgcctcgctcgagggtaagcccgag | |
| | ggcaagctggtgctcgtcaccgcgatctcgccgacgcctgcgggcgagggca | |
| | agaccaccacgactgtggggctcggcgacgcgctcaaccgcatcggcaagcg | |
| | ggcggtgatgtgcctgcgcgagccctcgctcggcccctgcttcggcatgaagg | |
| | gcggcgcggccggtggcggcaaggcgcaggtcgtgccgatggagcagatca | |
| | acctgcacttcaccggcgacttccacgccatcaccteggcgcactogctcgccg | |
| | ccgctctgatcgacaaccacatctactgggccaacgagctcaacatcgacgtgc | |
| | gccgcatccactggcgccgcgtggtcgacatgaacgaccgagcgctgcgcgc | |
| | gatcaaccagtcgctcggcggcgtcgccaacggctttccgcgtgaggacggctt | |
| | cgacatcaccgtcgcctccgaggtgatggcggtgttctgcctcgccaagaatctg | |
| | gctgacctcgaagagcggctcggccgcategtcatcgccgagaccegcgacc | |
| | gcaagccggtgacgctggccgacgtgaaggcgaccggtgcgatgaccgttctc | |
| | ctcaaggacgcgcttcagccgaacctcgtgcagacgctggagggcaacccgg | |
| | ccctgatccatggcggcccgttcgccaacategcccacggctgcaactcggtga | |
| | tcgccacccgcaccggcctgcggctggccgactacaccgtcaccgaggccgg | |
| | cttcggcgcggatctcggcgcggagaagttcatcgacatcaagtgccgccaga | |
| | ccggcctcaagccctcgtcggtggtgatcgtcgccacgatccgcgccctcaaga | |
| | tgcatggcggcgtcaacaagaaggatctccaggctgagaacctcgacgcgctg | |
| | gagaagggcttcgccaacctcgagcgccacgtgaataacgtccggagcttcgg | |
| | cctgccggtggtggtgggtgtgaaccacttcttccaggacaccgacgccgagca | |
| | tgcccggttgaaggagctgtgccgcgaccggctccaggtcgaggcgatcacct | |
| | gcaagcactgggcggagggcggcgcgggcgccgaggcgctggcgcaggcc | |
| | gtggtgaagctcgccgagggcgagcagaagccgctgaccttcgcctacgaga | |
| | ccgagacgaagatcaccgacaagatcaaggcgatcgcgaccaagctctacgg | |
| | cgcggccgacatccagatcgagtcgaaggccgccaccaagctcgccggcttc | |
| | gagaaagacggctacggcaaattgccggtctgcatggccaagacgcagtactc | |
| | gttctcgaccgacccgaccctgatgggcgcgccctcgggccacctcgtctcggt | |
| | gcgcgacgtgcgcctctcggcgggcgccggcttcgtcgtggtgatctgcggtg | |
| | agatcatgaccatgccgggtctgccaaaagtgccggcggcggacaccatccgc | |
| | ctggacgccaacggtcagatcgacgggctgttctag-3′ | |
| fch | Methenyl tetrahydrofolate cyclohydrolase, CP001298 | Methylobacterium |
| (SEQ ID NO: | REGION: 2231946-2232572 [SEQ ID NO: 2] | extorquens CM4 |
| 2) | 5′-atggccggcaacgagacgatcgaaacattcctcgacggcctggcgagctcggc | |
| | cccgacccccggcggcggcggtgccgccgcgatctccggcgccatgggcgc | |
| | ggcgctggtgtcgatggtgtgcaacctcaccatcggcaagaagaagtatgtcga | |
| | ggtcgaggccgacctgaagcaggtgctggagaagtcggaaggcctgcgccgc | |
| | acgctcaccggcatgatcgccgacgacgtcgaggctttcgacgcggtgatggg | |
| | cgcctacgggctgccgaagaacaccgacgaagagaaggccgcccgcgccgc | |
| | caagattcaagaggcgctcaaaaccgcgaccgacgtgccgctcgcctgctgcc | |
| | gcgtctgccgcgaggtgatcgacctggccgagatcgtcgccgagaagggcaat | |
| | ctcaacgtcatctcggatgccggcgtcgccgtgctctcggcctatgccggcctgc | |
| | gctcggcggcccttaacgtctacgtcaacgccaagggcctcgacgaccgcgcc | |
| | ttcgccgaggagcggctgaaggagctggagggcctactggccgaggcgggc | |
| | gcgctcaacgagcggatctacgagaccgtgaagtccaaggtaaactga-3′ | |
| mtdA | Methylene-tetrahydrofolate dehydrogenase, CP001298 | Methylobacterium |
| (SEQ ID NO: | REGION: 2230986-2231852 | extorquens CM4 |
| 3) | [SEQ ID NO: 3] 5′- | |
| | atgtccaagaagctgctcttccagttcgacaccgatgccacgccgagcgtcttcg | |
| | acgtcgtcgtcggctacgacggcggtgccgaccacatcaccggctacggcaac | |
| | gtcacgcccgacaacgtcggcgcctatgtcgacggcacgatctacacccgcgg | |
| | cggcaaggagaagcagtcgacggcgatcttcgtcggcggcggcgacatggcg | |
| | gccggcgagcgggtgttcgaggcggtgaagaagcgcttcttcggcccgttccg | |
| | cgtgtcctgcatgctggattcgaacggctccaacacgaccgctgcggcgggcgt | |
| | ggcgctcgtcgtcaaggcggcgggcggctcggtcaagggcaagaaggccgtc | |
| | gtgctcgcgggcaccggcccggtcggcatgcgctcggcggcgctgcttgccg | |
| | gcgagggcgccgaggtcgtgctgtgcgggcgcaagctcgacaaggcgcagg | |
| | ccgcggccgattccgtgaacaagcgcttcaaggtgaacgtcaccgcggccgag | |
| | accgcggacgacgcttcgcgtgccgaggccgtgaagggcgcccatttcgtcttc | |
| | accgccggtgcgatcggccttgaactgctgccgcaggcagcctggcagaacga | |
| | gagttcgatcgagatcgtggccgactacaacgcccagccgccgctcggcatcg | |
| | gcgggatcgatgcgaccgacaaaggcaaggaatacggcggaaagcgcgcctt | |
| | cggtgcgctcggcatcggcggcttgaagctcaagctgcaccgcgcctgcatcg | |
| | ccaagctgttcgagtcgagcgaaggcgtcttcgacgccgaggagatctacaag | |
| | ctggccaaggaaatggcctga-3′ | |
| fdh | Formate dehydrogenase, EC:1.17.1.9 [SEQ ID NO: 4] | Candida |
| (SEQ ID NO: | 5′- | boidinii |
| 4) | atgaagatcgttttagtcttatatgatgctggtaagcacgctgctgatgaagaaaaa | |
| | ttatatggttgtactgaaaataaattaggtattgctaattggttaaaagatcaaggtc | |
| | atgaactaattactacttctgataaagaaggtgaaacaagtgaattggataaacata | |
| | tcccagatgctgatattatcatcaccactcctttccatcctgcttatatcactaagga | |
| | aagacttgacaaggctaagaacttaaaattagtcgttgtcgctggtgttggttctga | |
| | tcacattgatttagattatattaatcaaacaggtaagaaaatctcagtcttggaagtt | |
| | acaggttctaatgttgtctctgttgctgaacacgttgtcatgaccatgcttgtcttggt | |
| | tagaaatttcgttccagcacatgaacaaattattaaccacgattgggaggttgctgc | |
| | tatcgctaaggatgcttacgatatcgaaggtaaaactattgctaccattggtgctgg | |
| | tagaattggttacagagtcttggaaagattactcccttttaatccaaaagaattattat | |
| | actacgattatcaagctttaccaaaagaagctgaagaaaaagttggtgctagaag | |
| | agttgaaaatattgaagaattagttgctcaagctgatatcgttacagttaatgctcca | |
| | ttacacgcaggtacaaaaggtttaattaataaggaattattatctaaatttaaaaaag | |
| | gtgcttggttagtcaataccgcaagaggtgctatttgtgttgctgaagatgttgcag | |
| | cagctttagaatctggtcaattaagaggttacggtggtgatgtttggttcccacaac | |
| | cagctccaaaggatcacccatggagagatatgagaaataaatatggtgctggtaa | |
| | tgccatgactcctcactactctggtactactttagatgctcaaacaagatacgctga | |
| | aggtactaaaaatatcttggaatcattctttactggtaaatttgattacagaccacaa | |
| | gatattatcttattaaatggtgaatacgttactaaagcttacggtaaacacgataaga | |
| | aataa-3′ | |
| fdhmut | Formate dehydrogenase mutant, NCBI_GeneID:831330 | Arabidopsis |
| (SEQ ID NO: | [SEQ ID NO: 5] | thaliana |
| 5) | 5′- | |
| | atggcgatgagacaagccgctaaggcaacgatcagggcctgttcttcctcttcttc | |
| | ttcgggttacttcgctcgacgtcagtttaatgcatcttctggtgatagcaaaaagatt | |
| | gtaggagttttctacaaggccaacgaatacgctaccaagaaccctaacttccttgg | |
| | ctgcgtcgagaatgccttaggaatccgtgactggcttgaatcccaaggacatcag | |
| | tacatcgtcactgatgacaaggaaggccctgattgcgaacttgagaaacatatcc | |
| | cggatcttcacgtcctaatctccactcccttccacccggcgtatgtaactgctgaaa | |
| | gaatcaagaaagccaaaaacttgaagcttctcctcacagctggtattggctcggat | |
| | catattgatctccaggcagctgcagctgctggcctgacggttgctgaagtcacgg | |
| | gaagcaacgtggtctcagtggcagaagatgagctcatgagaatcttaatcctcat | |
| | gcgcaacttcgtaccagggtacaaccaggtcgtcaaaggcgagtggaacgtcg | |
| | cgggcattgcgtacagagcttatgatcttgaagggaagacgataggaaccgtgg | |
| | gagctggaagaatcggaaagcttttgctgcagcggttgaaaccattcgggtgtaa | |
| | cttgttgtaccatcaaaggcatcagatggcaccagagctggagaaagagactgg | |
| | agctaagttcgttgaggatctgaatgaaatgctccctaaatgtgacgttatagtcat | |
| | caacatgcctctcacggagaagacaagaggaatgttcaacaaagagttgatagg | |
| | gaaattgaagaaaggcgttttgatagtgaacaacgcaagaggagccatcatgga | |
| | gaggcaagcagtggtggatgcggtggagagtggacacattggagggtacagc | |
| | ggagacgtttgggacccacagccagctcctaaggaccatccatggcgttacatg | |
| | cctaaccaggctatgacccctcatacctccggcaccaccattgacgctcagctac | |
| | ggtatgcggcggggacgaaagacatgttggagagatacttcaagggagaagac | |
| | ttccctactgagaattacatcgtcaaggacggtgaacttgctcctcagtaccggta | |
| | a-3′ | |
| | L229H mutant | |

The gene encoding at least one selected from the group consisting of formate-tetrahydrofolate ligase, methenyl tetrahydrofolate cyclohydrolase, methylene-tetrahydrofolate dehydrogenase, formate dehydrogenase, and formate dehydrogenase mutant may be introduced by cloning into a vector including a replication origin having a copy number of 1 to 12, and may preferably be introduced by cloning into a vector including a replication origin having a copy number of 1 to 5, but is not limited thereto.

The host microorganism may be selected from the group consisting of the genus Escherichia, the genus Mannheimia, the genus Rhodobacter, and the genus Methylobacterium, but is not limited thereto.

The recombinant microorganism according to the present invention produces lactic acid from formic acid and carbon dioxide using only formic acid and carbon dioxide as carbon sources.

The genes of the present invention may be altered in various ways in the coding region, so long as the amino acid sequence of the protein expressed from the coding region is not changed, and may be freely varied or modified so long as the expression of genes in regions excluding the coding region is not affected, and such varied or modified genes also fall within the scope of the present invention.

Therefore, the present invention also includes a polynucleotide having a nucleotide sequence substantially identical to the gene as well as a fragment of the gene. The term “substantially identical polynucleotide” means a gene encoding an enzyme having the same function as that used in the present invention, regardless of the homology of the sequence. The term “fragment of the gene” also means a gene encoding an enzyme having the same function as that used in the present invention, regardless of the length of the fragment.

In addition, the amino acid sequence of the protein, which is an expression product of the gene of the present invention, can be obtained from biological resources such as various microorganisms, so long as the titer and activity of the corresponding enzyme are not affected, and these biological resources also fall within the scope of the present invention.

Thus, the present invention also includes polypeptides having amino acid sequences substantially identical to the protein, and fragments of the polypeptides. The term “substantially identical polypeptide” means a protein having the same function as that used in the present invention, regardless of the homology of the amino acid sequence. The term “fragment of the polypeptide” also means a protein having the same function as that used in the present invention, regardless of the length of the fragment.

As used herein, the term “vector” means a DNA product containing a DNA sequence operably linked to a regulation sequence capable of expressing DNA in a suitable host, and may be a plasmid, a phage particle, or a simple potential genome insert. Once the vector is transformed into an appropriate host, it may replicate and function independently of the genome of the host, or may in some cases be integrated with the host genome. Since the plasmid is the most commonly used type of vector, the terms “plasmid” and “vector” are sometimes used interchangeably throughout the specification of the present invention. For the purpose of the present invention, a plasmid vector is preferably used. A typical plasmid vector that can be used for this purpose includes (a) a replication origin to efficiently conduct replication so as to include several to several hundred plasmid vectors per host cell, (b) an antibiotic resistance gene to screen a host cell transformed with the plasmid vector, and (C) a restriction enzyme cleavage site into which a foreign DNA fragment is inserted. Even if an appropriate restriction enzyme cleavage site is not present, the vector and foreign DNA can be easily ligated using a synthetic oligonucleotide adapter or a linker according to a conventional method. After ligation, the vector should be transformed into an appropriate host cell. Transformation can be easily carried out using a calcium chloride or electroporation method (Neumann, et al., EMBO J., 1: 841, 1982).

In the present invention, “gene is reduced (or inactivated)” means that expression of the gene is reduced compared to the wild type, or that the function or activity of the protein encoded by the gene is reduced compared to the wild type.

Expression vectors well-known in the art can be used as vectors for enhancing or overexpressing genes according to the present invention.

When a nucleotide sequence is aligned with another nucleotide sequence based on a functional relationship, it is said to be “operably linked” thereto. This may be gene(s) and regulatory sequence(s) linked in such a way so as to enable gene expression when a suitable molecule (e.g., a transcriptional activator protein) is linked to the regulatory sequence(s). For example, DNA for a pre-sequence or secretory leader is operably linked to DNA for a polypeptide when expressed as a pre-protein involved in secretion of the polypeptide, a promoter or enhancer is operably linked to a coding sequence when it affects the transcription of the sequence, a ribosome-binding site is operably linked to a coding sequence when it affects the transcription of the sequence, or a ribosome-binding site is operably linked to a coding sequence when positioned to facilitate translation. Generally, “operably linked” means that the linked DNA sequence is in contact therewith, or that a secretory leader is in contact therewith and is present in the reading frame. However, the enhancer need not be in contact. The linkage of these sequences is carried out by ligation (linkage) at a convenient restriction enzyme site. When no such site exists, a synthetic oligonucleotide adapter or a linker according to a conventional method is used.

As is well known in the art, in order to increase the expression level of a transgene in a host cell, the gene should be operably linked to a transcriptional/translational expression regulation sequence that functions in a selected expression host. Preferably, the expression regulation sequence and the corresponding gene are included in one recombinant vector containing both a bacterial selection marker and a replication origin. When the host cell is a eukaryotic cell, the recombinant vector should further include a useful expression marker in the eukaryotic expression host.

The host cell transformed with the recombinant vector described above constitutes another aspect of the present invention. As used herein, the term “transformation” means introducing DNA into a host and making the DNA replicable using an extrachromosomal factor or chromosomal integration.

It should be understood that not all vectors function identically in expressing the DNA sequences of the present invention. Likewise, not all hosts function identically for the same expression system. However, those skilled in the art will be able to make appropriate selections from among a variety of vectors, expression regulation sequences, and hosts without excessive burden of experimentation and without departing from the scope of the present invention. For example, selection of a vector should be carried out in consideration of a host because the vector should be replicated therein. The number of replications of the vector, the ability to control the number of replications, and the expression of other proteins encoded by the corresponding vector, such as the expression of antibiotic markers, should also be considered.

In addition, the gene introduced in the present invention may be introduced into the genome of a host cell, and may exist as a chromosomal factor. It will be apparent to those skilled in the art that even insertion of the gene into the genome of the host cell has the same effect as introducing the recombinant vector into the host cell.

The recombinant microorganism described above can produce lactic acid from pyruvate assimilated from formic acid and carbon dioxide.

The host microorganism may be selected from, but is not limited to, the group consisting of, for example, Escherichia, Mannheimia, Rhodobacter, and Methylobacterium.

Escherichia coli was transformed with the produced recombinant plasmid to produce a recombinant E. coli. The E. coli strain used in the present invention was E. coli DH5a (Invitrogen, USA), and transformation into E. coli was performed using chemical transformation commonly used in the art.

Meanwhile, the present invention showed that the recombinant microorganism could produce 4.39 g/L and 25.5 mg/L of lactic acid, respectively, under conditions of carbon dioxide, formic acid, and glucose, and in a culture medium providing only carbon dioxide and formic acid as carbon sources.

Therefore, in another aspect, the present invention is directed to a method of producing a beneficial substance, the method:
(a) culturing the recombinant microorganism using formic acid and carbon dioxide as carbon sources to produce a beneficial substance; and
(b) recovering the produced beneficial substance.

In the present invention, the beneficial substance may be lactic acid.

In yet another aspect, the present invention is directed to a method of producing lactic acid from carbon dioxide using a bioelectric hybrid system, the method:
(a) converting carbon dioxide into formic acid through an electrochemical reduction reaction; and
(b) fermenting a microorganism capable of biosynthesizing lactic acid from formic acid and carbon dioxide using the formic acid converted in step (a), to produce lactic acid.

In the present invention, in step (a), carbon dioxide is converted into formic acid by an electrochemical reduction reaction at a negative electrode, and in step (b), the fermentation is performed in the presence of an electrode solution containing formic acid converted in step (a), and the electrode solution may be a negative electrode solution.

In the present invention, the concentration of formic acid contained in the negative electrode solution may be 0.1 to 10 g/L.

In the present invention, the electrode solution may contain a phosphoric acid compound and the concentration of the phosphoric acid compound in the electrode solution may be 10 to 40 g/L.

In the present invention, the phosphoric acid compound may include at least one selected from the group consisting of potassium dihydrogen phosphate (KH₂PO₄), dipotassium hydrogen phosphate (K₂HPO₄), and tripotassium phosphate (K₃PO₄).

In the present invention, the microorganism capable of biosynthesizing lactic acid from carbon dioxide and formic acid may be the recombinant microorganism.

In another aspect, the present invention is directed to a bioelectric hybrid system device for producing lactic acid from carbon dioxide, including:
(i) an electrolysis reactor for converting carbon dioxide into formic acid by an electrochemical reduction reaction; and
(ii) a biosynthesis reactor fluidly connected to the electrolysis reactor and producing lactic acid from formic acid through fermentation of a microorganism in an electrode solution containing formic acid.
(a) of FIG. 4 is a schematic diagram illustrating a bioelectric hybrid system device utilizing electrochemical carbon dioxide reduction and microbial fermentation according to an embodiment of the present invention.

The bioelectric hybrid system device may further include a first conduit, which fluidly connects the electrolysis reactor to the biosynthesis reactor and transports the negative electrode solution containing formic acid within the electrolysis reactor to the biosynthesis reactor.

Furthermore, the bioelectric hybrid system device may further include a second conduit, which fluidly connects the biosynthesis reactor to the electrolysis reactor and transports the electrode solution within the biosynthesis reactor to the electrolysis reactor.

The electrolysis reactor may include a gas chamber, a negative electrode solution chamber, a positive electrode solution chamber, a gas diffusion electrode, a cation exchange membrane, and a catalyst. Specifically, the electrolysis reactor may include the gas diffusion electrode provided between the gas chamber and the cathode electrode solution chamber, and the cation exchange membrane provided between the negative electrode solution chamber and the positive electrode solution chamber. Furthermore, the negative electrode solution chamber may include a reference electrode and a counter electrode may be installed outside the positive electrode solution chamber.

The gas chamber can serve as a channel through which carbon dioxide gas flows to the gas diffusion electrode, the negative electrode solution chamber can serve as a channel through which a negative electrode solution flows, and the positive electrode solution chamber can serve as a channel through which the positive electrode solution flows.

The gas diffusion electrode is a porous electrode that ensures gas permeability to facilitate gas-related electrode reactions by allowing the three phases of the electrode (solid), electrode solution (liquid), and gas to contact each other. The gas diffusion electrode may be a water-repellent electrode to prevent electrode solution overfilling and may serve as both a negative electrode and a working electrode in the electrolysis reactor. Furthermore, the gas diffusion electrode may provide gaseous carbon dioxide near the active site of the electrocatalyst, thus facilitating high current density operation of the electrochemical carbon dioxide reaction.

At the positive electrode, a reaction occurs where water decomposes to produce hydrogen ions and oxygen gas. The hydrogen ions generated at the positive electrode may migrate through the cation exchange membrane to the gas diffusion electrode and be supplied to the gas diffusion electrode for the formic acid production reaction.

At the gas diffusion electrode, which functions as a negative electrode, carbon dioxide supplied from the gas chamber to the gas diffusion electrode may be converted into formic acid through an electrochemical reduction reaction with hydrogen ions generated by water decomposition at the positive electrode. The negative electrode solution containing formic acid converted in this way may be introduced from the electrolysis reactor to the biosynthesis reactor through the first conduit and may serve as a nutrient for lactic acid synthesis by microorganisms present in the biosynthesis reactor.

The electrochemical reduction to formic acid exhibits high selectivity under environmentally favorable reaction conditions, maximizing carbon dioxide utilization and providing an environmentally beneficial approach.

The concentration of formic acid in the negative electrode solution may range from 0.1 to 10 g/L, preferably from 0.3 to 6 g/L, and more preferably from 0.5 to 3 g/L. When the concentration of formic acid falls below this range, microorganisms may not easily access carbon sources and instead digest lactic acid instead of formic acid. When the concentration exceeds this range, the toxicity of formic acid may affect microbial metabolism.

The cation exchange membrane reduces the crossover of organic substances, such as substrates that serve as nutrients for microorganisms at the positive electrode, thereby preventing contamination of the negative electrode by organic substances. Furthermore, the cation exchange membrane reduces the permeability of metal cations and enhances the permeability of hydrogen ions, thereby preventing a decrease in the pH of the positive electrode and improving the efficiency of the electrolysis system while maintaining stable efficiency.

The catalyst may be a precious metal catalyst, a non-precious metal catalyst, or an organometallic catalyst. Alternatively, a catalyst structure in which these catalysts are dispersed on various supports may be used. Examples of precious metal catalysts include Pt, Ru, Ir, Rh, Pd, Au, Ag, and Re, and examples of non-precious metal catalysts include Fe, Co, Ni, Mn, Mo, Zn, V, Cr, Cu, Al, Ga, Ge, In, Sn, and Sb. A representative example of the non-precious metal catalyst is Sn. Because Sn is abundant on Earth and is non-toxic, Sn catalysts may be used in CO₂ reduction reactions.

Examples of organometallic catalysts include Fe-based organometallics, Co-based organometallics, Ni-based organometallics, Mn-based organometallics, Cu-based organometallics, and Sn-based organometallics. These catalysts may be used alone or as an alloy. For example, alloy-type organometallic catalysts include precious metal-based binary alloy catalysts, platinum-transition metal-based binary alloy catalysts, platinum-organometallic binary alloy catalysts, or multi-component alloy catalysts.

These metal catalysts may be used on their own or supported on a support. These supports may be carbon-based materials such as graphite, Denka black, Ketjen black, acetylene black, carbon nanotubes, carbon nanofibers, carbon nanowires, carbon nanoballs, or activated carbon. Alternatively, the supports may be inorganic particles such as alumina, silica, zirconia, or titania. However, carbon-based materials are generally used.

Next, the biosynthesis reactor contains microorganisms that synthesize lactic acid and an electrode solution for microbial growth.

In an embodiment of the present invention, the electrode solution in which the microorganism can survive contains 2.1 g/L KH₂PO₄, 39 g/L K₂HPO₄, 0.5 g/L MgSO₄, 0.8 g/L citric acid, 0.012 g/L IPTG, and 2 g/L NH₄Cl, but is not limited thereto.

The hollow fiber filter may be disposed between the electrolysis reactor and the biosynthesis reactor to separate the microorganism from the electrode solution before the electrode solution in the biosynthesis reactor is introduced into the electrolysis reactor.

Furthermore, step (b) may include biosynthesizing lactic acid through a microbial fermentation process in the electrode solution mixed with the negative electrode solution containing formic acid in step (a). The initial inoculation amount of microorganisms may be a cell density of OD₆₀₀ 2 to OD₆₀₀ 10, but is not limited thereto.

Furthermore, in step (b), a current density to produce formic acid through electrochemical carbon dioxide reduction in the electrochemical electrolyzer may be −90 mA cm⁻² to −140 mA cm⁻², but is not limited thereto.

The recombinant microorganism according to the present invention can produce lactic acid using only carbon dioxide and formic acid. The recombinant microorganism developed by the present inventors in a previous study was capable of growing solely on carbon dioxide and formic acid without glucose supply, but was capable of producing lactic acid. However, the recombinant microorganism according to the present invention was capable of producing 25 mg/L of lactic acid solely from carbon dioxide and formic acid by removing the endogenous lactate dehydrogenase genes and introducing a gene encoding a heterologous lactate dehydrogenase.

Furthermore, in the present invention, the bioelectric hybrid system produced based on combination of the method for producing lactic acid solely from formic acid and carbon dioxide with the electrode system for producing formic acid from carbon dioxide was capable of producing 88.3 mg/L of lactic acid solely from carbon dioxide. This was achieved through optimization of the developed electrode solution, initial microbial inoculation amount, and current density.

### EXAMPLE

Hereinafter, the present invention will be described in more detail with reference to the following examples. However, it will be obvious to those skilled in the art that the following examples are provided only for illustration of the present invention and should not be construed as limiting the scope of the present invention.

### Example 1: Development of Recombinant Microorganism Producing Lactic Acid from Carbon Dioxide and Formic Acid Through Improvement of Metabolically Engineered Strain

To develop a strain with enhanced assimilation efficiency from carbon dioxide and formic acid alone, the genes encoding lactate dehydrogenase (ldhA, dld, and lldD) from Escherichia coli were deleted. A heterologous lactate dehydrogenase (ldhₛₜᵣₑₚ) gene derived from Streptococcus was introduced to develop a recombinant microorganism capable of selectively producing L-lactic acid.

Specifically, PCR was performed using the genomic DNA of a microorganism having the heterologous lactate dehydrogenase (ldhₛₜᵣₑₚ) gene among the gene fragments required for plasmid construction for foreign gene introduction as a template and primers designed for amplification of the gene, and then the amplified gene fragments were recovered and purified. The sequences of the genes and primers are shown in Tables 2 and 3.

TABLE 2

| Target | | Origin |
|---|---|---|
| gene | NCBI information | microorganism |
| 1dhₛₜᵣₑₚ | L-lactate dehydrogenase NCBI | Streptococcus |
| (SEQ ID | Sequence ID: ARC49562.1\| | |
| NO: 6) | [SEQ ID NO: 06: 5′- | |
| | atgaccgctactaagcagcacaagaaggta | |
| | attctggtaggtgacggtgcagttggttct | |
| | agttacgcctttgcgctggttaatcaaggc | |
| | atcgcgcaggaactgggtatcatcgagatt | |
| | ccccagctttttgagaaagccgtcggggat | |
| | gcgcttgacttgtcccatgcgttagctttc | |
| | acctcaccgaaaaagatctacgcggcaaaa | |
| | tacgaggattgcgccgatgcagatttagtc | |
| | gtaatcacagcaggggcgccgcagaagccc | |
| | ggcgagactcgtctggacctggtcgggaaa | |
| | aatttggccattaacaaatcaatcgtcacc | |
| | caagtcgttgaatcgggattcaacggtata | |
| | tttcttgtagccgcaaaccccgttgacgta | |
| | ttaacttattcgacatggaaatttagtggg | |
| | tttccaaaggaaagagtcataggttctgga | |
| | acttctcttgatagcgcgagatttcgtcaa | |
| | gcactggctgagaagttaggcgttgatgct | |
| | cgctccgttcatgcctacataatgggcgaa | |
| | catggggactctgaatttgcagtttggtcc | |
| | cacgctaacatagctggtgtcaaccttgaa | |
| | gaattcctgaaagatacgcagaatgtccag | |
| | gagagtgagttggtcgaattattcgaggga | |
| | gtgcgtgacgccgcttacacgattattaat | |
| | aaaaaaggggctacctattacggcattgca | |
| | gttgcgcttgcccgtatcaccaaagcaatc | |
| | cttgacgatgagaatgcagttttgcctctt | |
| | agcgtatttcaggaaggacaatacggagtg | |
| | agtaacgtcttcataggacaaccggcaatc | |
| | gtgggagcacacggaattgttcgtcccgtt | |
| | aatataccgcttaatgatgcggaacagcaa | |
| | aagatgaaagcttcggctgatgagctgcag | |
| | gccataatcgacgaagcctggaaaaaccca | |
| | gaattccaagaggcttccaagaattaa | |
| | -3′ | |

TABLE 3

| Target template | Primer sequence |
|---|---|
| 1dhₛₜᵣₑₚ | [SEQ ID NO: 7]: |
| (SEQ ID | 5′-TTGCTCCTCAGTACCGGTAAA |
| NO: 6) | GGAGGAATTCATGACCGCTACTAA |
| | GCAGCA-3′ |
| | [SEQ ID NO: 8]: 5′- |
| | CATCCGCCAAAACAGCCAAGTTAA |
| | TTCTTGGAAGCCTCTTGGAA-3′ |
| p518FA1 | [SEQ ID NO: 9]: |
| | 5′-TTGCTCCTCAGTACCGGTAA-3′ |
| | [SEQ ID NO: 10]: |
| | 5′-TTAATTCTTGGAAGCCTCTTGGA-3′ |

The plasmid backbone gene fragment was prepared by performing PCR using the primers, followed by recovery and purification of the amplified gene fragment.

The plasmid backbone gene fragment and the amplified gene fragment were assembled into plasmids using the Gibson assembly method (Gibson et al., Nat. Methods, 6:5, 343-345, 2009), a commonly used method for assembling gene fragments. Each plasmid was constructed to contain one or more of the foreign genes listed in the table provided above. (FIG. 2)

TABLE 4

| Plasmid | Characteristics |
|---|---|
| p518FA1 | Plasmid including p15A replication origin, chloramphenicol |
| | resistance gene, BBa_23100 synthetic formate-tetrahydrofolate |
| | ligase, methenyl tetrahydrofolate cyclohydrolase, methylene- |
| | tetrahydrofolate dehydrogenase, formate dehydrogenase, and |
| | formate dehydrogenase mutant. |
| p518FA1R_Strepto_ldhA | Plasmid including L-lactate dehydrogenase (SEQ ID NO: 6) in |
| | addition to p518FA1 plasmid. |

Escherichia coli was transformed with the recombinant plasmid produced by the method to produce recombinant E. coli The E. coli strain used in the present invention was E. coli DH5α (Invitrogen, USA), and transformation was performed using a chemical transformation method commonly used in the art.

In addition, the endogenous lactate dehydrogenase genes ldhA, dld, and lldD present in E. coli were deleted.

TABLE 5

| Strain | |
|---|---|
| name | Genotype |
| DH5α | F- Φ80lacZΔM15 Δ(lacZYA-argF) U169 recA1 endA1 |
| | hsdR17(rk−, mk+) phoA supE44 thi⁻¹ gyrA96 relA1 λ− |
| AKO | purT, ppsR gene was knocked out and native promoter |
| | of ppsA gene was changed to trc promoter in the |
| | DH5α_GTPS strain |
| AKOd | dld gene was knocked out from the AKO1 strain |
| AKOdldhA | Native promoter of ldhA gene was replaced with trc |
| | promoter from the AKOd strain |
| AKOldl | ldhA and lldD genes were knocked out from the AKOd |
| | strain |

The following strains were produced by introducing the p518FA1R_Strepto_ldhA plasmid into the strains produced above.

TABLE 6

| Strain | |
|---|---|
| name | Genotype |
| AKO1 | ppsR gene was knocked out and native promoter of |
| | ppsA gene was changed to trc promoter in the |
| | DH5α_GTPS strain |
| FC15 | AKOldl harboring p518strep |

In order to determine the enhanced carbon dioxide and formic acid assimilation capacity of the recombinant E. coli strains, the recombinant E. coli strains as an experimental group and the previously established E. coli strains acting and a control group were cultured in flasks in m9 medium containing formic acid and carbon dioxide. The m9 medium has the following composition (Table 7).

TABLE 7

| Ingredient | Content | |
|---|---|---|
| Sodium formate | 4.4 | g/L |
| NaHCO₃ | 4.2 | g/L |
| EDTA | 0.058 | g/L |
| NH₄Cl | 1.0 | g/L |
| MgSO₄•7H₂O | 0.4928 | g/L |
| Anti (CM) | 0.017 | g/L |
| IPTG | 0.012 | g/L |
| Thiamine | 0.005 | g/L |
| Na₂HPO₄ | 18.0 | g/L |
| KH₂PO₄ | 15.0 | g/L |
| NaCl | 2.5 | g/L |
| NH₄Cl | 5.0 | g/L |
| FeSO₄•7H₂O | 0.0500 | g/L |
| ZnSO₄•7H₂O | 0.0113 | g/L |
| CuSO₄•5H₂O | 0.0050 | g/L |
| MnSO₄•5H₂O | 0.0025 | g/L |
| Na₂B₄O₇•10H₂O | 0.0012 | g/L |
| CaCl₂•2H₂O | 0.0100 | g/L |
| (NH₄)₆Mo₇O₂₄ | 0.0005 | g/L |

| | |
|---|---|
| 5M HCl | 0.25M |

To determine production of lactic acid from the produced FC15 strain, the FC15 strain was first subjected to flask culture in an MM9 medium containing glucose, and to fermentor culture in an MM9 medium in which formic acid and carbon dioxide were provided as carbon sources. In the glucose-containing medium, 300 μL of a 10% formic acid solution was further supplied to supplement formic acid. Under the culture conditions in which glucose, formic acid, and carbon dioxide were provided, the FC15 strain produced 4.39 g/L of lactic acid ((b) of FIG. 1). Under the fermentor culture conditions in which only carbon dioxide and formic acid were provided as carbon sources, the FC15 strain produced 25.5 mg/L of lactic acid ((c) of FIG. 1). To evaluate lactic acid production capability of the parental strain AKO1, fermentor-level culture was performed in which only carbon dioxide and formic acid were supplied as carbon sources. Under an initial inoculum condition of OD₆₀₀=4.03, 143.5 mg/L of lactic acid was produced, whereas no lactic acid was detected in the AKO1 strain ((a) and (b) of FIG. 3).

Furthermore, the ¹³C labeling ratio of lactic acid was determined under conditions where ¹³C radiolabeled glucose was provided as a carbon source in the pre-fermentation step, and under conditions where only carbon dioxide and formic acid were provided as carbon sources without ¹³C radiolabeled glucose. Under conditions containing ¹³C-labeled glucose, approximately 88% of lactic acid was ¹³C-labeled. However, under conditions where only formic acid and carbon dioxide were provided as carbon sources, only approximately 10% of lactic acid was labeled, similar to the control group ((c) of FIG. 3). These results show that the FC15 strain can produce lactic acid solely from carbon dioxide and formic acid.

### Example 2: Development of Bioelectric Hybrid System for Producing Lactic Acid from Carbon Dioxide

A bioelectric hybrid system for producing lactic acid from carbon dioxide was developed by combining the lactic acid production method using the recombinant FC15 strain constructed in Example 1 with an electrode system for producing formic acid from carbon dioxide ((a) of FIG. 4).

In order to develop a bioelectric hybrid system, a physiologically compatible negative electrode solution capable of producing lactic acid was developed. A physiologically compatible negative electrode solution must have sufficient ionic conductivity to serve as an electrolyte and must be free of trace metal solutions to prevent the deposition of trace metals on the electrode surface. Furthermore, a nitrogen source must be excluded to prevent solid precipitation.

However, in the metabolic pathway of the FC15 strain, glycine serves as a precursor for lactic acid synthesis. Glycine synthesis requires a nitrogen source and glycine is subsequently degraded, leaving no nitrogen compounds consumed in the overall metabolic cycle ((a) FIG. 1). Therefore, a nitrogen source was required through the negative electrolyte. This was achieved by adding 2 g/L of NH₄Cl to the conventional biphosphate solution. The composition of the developed negative electrode solution is shown in Table 8.

TABLE 8

| Ingredient | Content | |
|---|---|---|
| NH₄Cl | 2.0 | g/L |
| MgSO₄•7H₂O | 0.5 | g/L |
| IPTG | 0.012 | g/L |
| K₂HPO₄ | 39.0 | g/L |
| KH₂PO₄ | 1.0 | g/L |
| Citric acid | 0.8 | g/L |

The CO₂ electrolyzer was operated at a high current density of −90 to −140 mA/cm² using the developed negative electrode solution, resulting in stable production of formic acid ((b) of FIG. 4).

The bioelectric hybrid system was operated using a Sn gas diffusion electrode and the FC15 strain. The CO₂ electrolyzer was first operated to accumulate formic acid. Then, the FC15 strain was inoculated into the fermenter and the hybrid system was operated for an additional 72 hours. t=0 h means the time point at which the FC15 strain was inoculated.

To determine the optimal cell density of the system, cells in the same growth state were inoculated at various initial cell densities. Experiments were conducted at −120 mA/cm² with initial OD₆₀₀ of 1.5 (1.74 mg dry cell weight), 3.3 (21.4 mg), and 5.0 (36.6 mg), respectively.

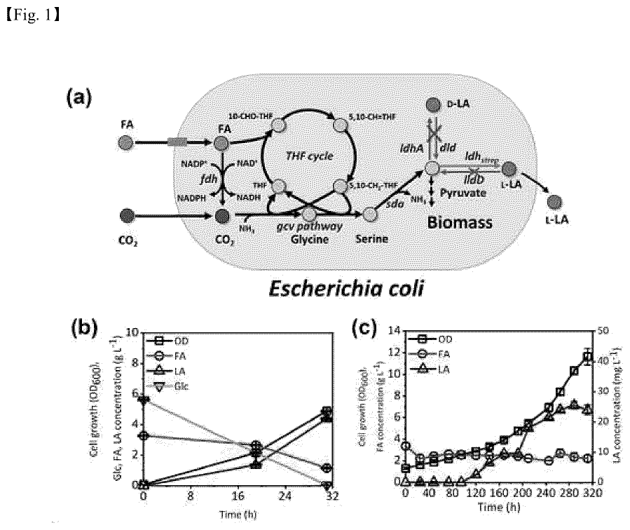

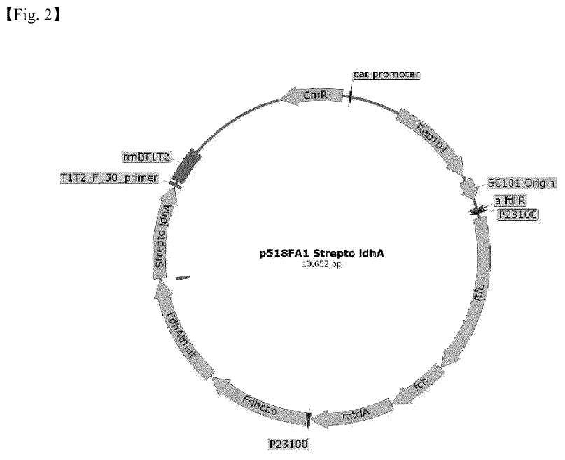

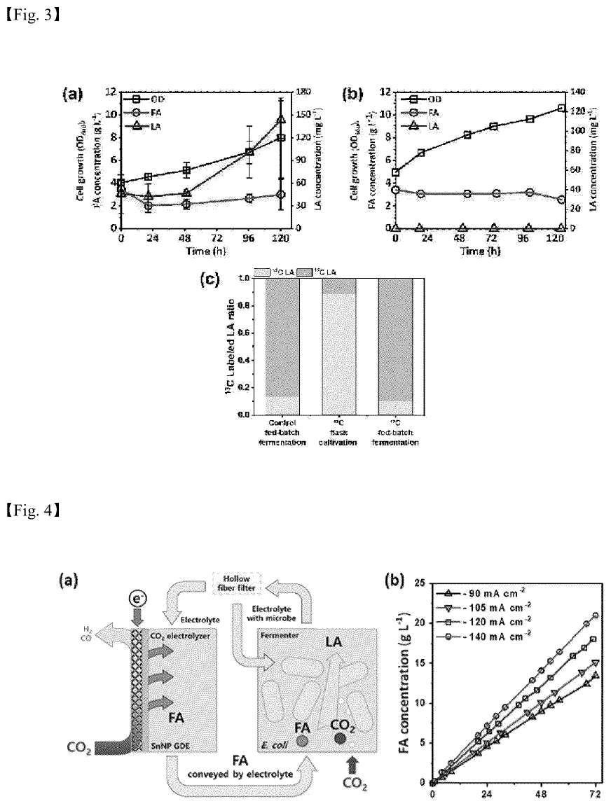

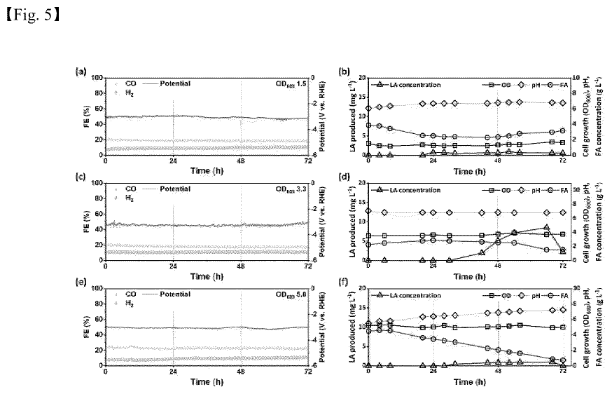

As shown in FIG. 5, formic acid produced through electrochemical CO₂ reduction was consumed by the FC15 strain under all conditions, with the highest lactic acid production observed under an OD₆₀₀ of 3.3.

Under an initial OD₆₀₀ of 3.3, lactic acid production was observed at various current densities (−90, −105, −120, and −140 mA cm²).

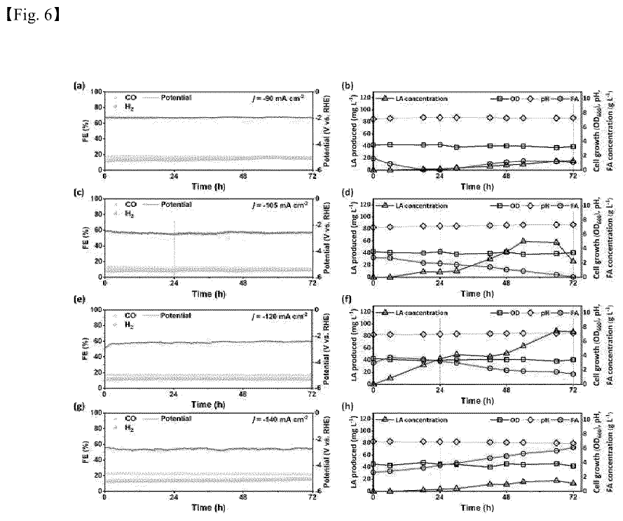

As shown in FIG. 6, changes in current density resulted in changes in formic acid supply, which affected lactic acid production. At a current density of −120 mA cm², a maximum of 88.3 mg/L of lactic acid was produced over 66 hours. The electrochemical reaction voltage and Faradaic efficiency remained stable under all conditions.

At a low current density of −90 mA/cm², formic acid was completely consumed in the early stages, and after 24 hours, the formic acid concentration decreased to 0, resulting in a low lactic acid production of 20 mg/L. At a current density of −105 mA/cm², a maximum of 68 mg/L of lactic acid was produced, but it was subsequently degraded as L-LA was used for cell metabolism. At a current density of −120 mA/cm², lactic acid was produced more stably, indicating that the supply of FA was sufficient to prevent the degradation of lactic acid. However, when the current density increased to −140 mA/cm², lactic acid production decreased to 22 mg L1, and formic acid concentration increased along with a decrease in pH. At the high concentration of formic acid, the cells could not efficiently convert formic acid into lactic acid due to the toxicity thereof.

Although specific configurations of the present invention have been described in detail, those skilled in the art will appreciate that this detailed description is provided as preferred embodiments for illustrative purposes and should not be construed as limiting the scope of the present invention. Therefore, the substantial scope of the present invention is defined by the accompanying filed claims and equivalents thereto.

What is claimed is:

1. A recombinant microorganism capable of producing lactic acid from carbon dioxide and formic acid,
wherein a gene encoding L-lactate dehydrogenase is introduced into or amplified in a host microorganism having a central carbon assimilation pathway.

2. The recombinant microorganism according to claim 1, wherein the host microorganism has i) an endogenous central carbon assimilation pathway or ii) an exogenous central carbon assimilation pathway.

3. The recombinant microorganism according to claim 1, wherein the gene encoding L-lactate dehydrogenase is represented by a base sequence of SEQ ID NO: 6.

4. The recombinant microorganism according to claim 1, wherein the host cell has no endogenous lactate dehydrogenase gene.

5. The recombinant microorganism according to claim 1, further comprising a gene encoding at least one selected from the group consisting of formate-tetrahydrofolate ligase, methenyl tetrahydrofolate cyclohydrolase, methylene-tetrahydrofolate dehydrogenase, formate dehydrogenase, and a formate dehydrogenase mutant.

6. The recombinant microorganism according to claim 5, wherein the gene encoding formate-tetrahydrophorate ligase is represented by a sequence of SEQ ID NO: 1,
the gene encoding methenyltetrahydrophorate cyclohydrolase is represented by a sequence of SEQ ID NO: 2,
the methylene-tetrahydrophorate dehydrogenase is represented by a sequence of SEQ ID NO: 3,
the formate dehydrogenase is represented by a sequence of SEQ ID NO: 4, or
the formate dehydrogenase mutant is represented by a sequence of SEQ ID NO: 5.

7. The recombinant microorganism according to claim 5, wherein the gene is introduced by cloning into a vector containing an origin of replication having a copy number of 1 to 5.

8. The recombinant microorganism according to claim 1, wherein the host microorganism is selected from the group consisting of the genus Escherichia, the genus Mannheimia, the genus Rhodobacter, and the genus Methylobacterium.

9. A method of producing a beneficial substance, including:
(a) culturing the recombinant microorganism according to claim 1 using formic acid and carbon dioxide as a carbon source to produce a beneficial substance; and
(b) recovering the produced beneficial substance.

10. The method according to claim 9, wherein the beneficial substance is lactic acid.

11. A method of producing lactic acid from carbon dioxide using a bioelectric hybrid system, the method including:
(a) converting carbon dioxide into formic acid through an electrochemical reduction reaction; and
(b) fermenting a microorganism capable of biosynthesizing lactic acid from formic acid and carbon dioxide using the formic acid converted in step (a), to produce lactic acid.

12. The method according to claim 11, wherein, in step (a), carbon dioxide is converted into formic acid by an electrochemical reduction reaction at a negative electrode.

13. The method according to claim 11, wherein, in step (b), the fermentation is performed in the presence of an electrode solution containing formic acid converted in step (a).

14. The method according to claim 13, wherein the electrode solution in step (b) is a negative electrode solution.

15. The method according to claim 14, wherein a concentration of formic acid contained in the negative electrode solution is 0.1 to 10 g/L.

16. The method according to claim 13, wherein the electrode solution contains a phosphoric acid compound.

17. The method according to claim 16, wherein a concentration of the phosphoric acid compound in the electrode solution is 10 to 40 g/L.

18. The method according to claim 16, wherein the phosphoric acid compound comprises at least one selected from the group consisting of potassium dihydrogen phosphate (KH₂PO₄), dipotassium hydrogen phosphate (K₂HPO₄), and tripotassium phosphate (K₃PO₄).

19. The method according to claim 11, wherein the microorganism capable of biosynthesizing lactic acid from carbon dioxide and formic acid is a recombinant microorganism capable of producing lactic acid from carbon dioxide and formic acid,
wherein a gene encoding L-lactate dehydrogenase is introduced into or amplified in a host microorganism having a central carbon assimilation pathway.

20. A bioelectric hybrid system device for producing lactic acid from carbon dioxide, including:
(i) an electrolysis reactor for converting carbon dioxide into formic acid by an electrochemical reduction reaction; and
(ii) a biosynthesis reactor fluidly connected to the electrolysis reactor and producing lactic acid from formic acid through fermentation of a microorganism in an electrode solution containing formic acid.

21. The bioelectric hybrid system device according to claim 20, wherein the electrolysis reactor comprises a gas diffusion electrode as a negative electrode.

22. The bioelectric hybrid system device according to claim 20, further comprising a first conduit to fluidly connect the electrolysis reactor to the biosynthesis reactor and transport the negative electrode solution containing formic acid within the electrolysis reactor to the biosynthesis reactor.

23. The bioelectric hybrid system device according to claim 20, further comprising a second conduit to fluidly connect the biosynthesis reactor to the electrolysis reactor and transport the electrode solution within the biosynthesis reactor to the electrolysis reactor.

24. The bioelectric hybrid system device according to claim 20, further comprising a hollow fiber filter to separate the microorganism from the electrode solution before the culture solution in the biosynthesis reactor is introduced into the electrolysis reactor.


SEQUENCE LISTING (10 sequences)

SEQ ID NO: 1 - DNA, 1674 bp, Methylobacterium extorquens
  source 1..1674: mol_type = genomic DNA; note = Methylobacterium extorquens CM4
atgccctcagatatcgagatcgcccgcgcggcgaccctgaagccgatcgcccaggtcgccgaaaagctcggcatcccggacgaggcgcttcacaattacggcaagcacatcgccaagatcgaccacgacttcatcgcctcgctcgagggtaagcccgagggcaagctggtgctcgtcaccgcgatctcgccgacgcctgcgggcgagggcaagaccaccacgactgtggggctcggcgacgcgctcaaccgcatcggcaagcgggcggtgatgtgcctgcgcgagccctcgctcggcccctgcttcggcatgaagggcggcgcggccggtggcggcaaggcgcaggtcgtgccgatggagcagatcaacctgcacttcaccggcgacttccacgccatcacctcggcgcactcgctcgccgccgctctgatcgacaaccacatctactgggccaacgagctcaacatcgacgtgcgccgcatccactggcgccgcgtggtcgacatgaacgaccgagcgctgcgcgcgatcaaccagtcgctcggcggcgtcgccaacggctttccgcgtgaggacggcttcgacatcaccgtcgcctccgaggtgatggcggtgttctgcctcgccaagaatctggctgacctcgaagagcggctcggccgcatcgtcatcgccgagacccgcgaccgcaagccggtgacgctggccgacgtgaaggcgaccggtgcgatgaccgttctcctcaaggacgcgcttcagccgaacctcgtgcagacgctggagggcaacccggccctgatccatggcggcccgttcgccaacatcgcccacggctgcaactcggtgatcgccacccgcaccggcctgcggctggccgactacaccgtcaccgaggccggcttcggcgcggatctcggcgcggagaagttcatcgacatcaagtgccgccagaccggcctcaagccctcgtcggtggtgatcgtcgccacgatccgcgccctcaagatgcatggcggcgtcaacaagaaggatctccaggctgagaacctcgacgcgctggagaagggcttcgccaacctcgagcgccacgtgaataacgtccggagcttcggcctgccggtggtggtgggtgtgaaccacttcttccaggacaccgacgccgagcatgcccggttgaaggagctgtgccgcgaccggctccaggtcgaggcgatcacctgcaagcactgggcggagggcggcgcgggcgccgaggcgctggcgcaggccgtggtgaagctcgccgagggcgagcagaagccgctgaccttcgcctacgagaccgagacgaagatcaccgacaagatcaaggcgatcgcgaccaagctctacggcgcggccgacatccagatcgagtcgaaggccgccaccaagctcgccggcttcgagaaagacggctacggcaaattgccggtctgcatggccaagacgcagtactcgttctcgaccgacccgaccctgatgggcgcgccctcgggccacctcgtctcggtgcgcgacgtgcgcctctcggcgggcgccggcttcgtcgtggtgatctgcggtgagatcatgaccatgccgggtctgccaaaagtgccggcggcggacaccatccgcctggacgccaacggtcagatcgacgggctgttctag

SEQ ID NO: 2 - DNA, 627 bp, Methylobacterium extorquens
  source 1..627: mol_type = genomic DNA
  misc_feature 1..627: note = Methylobacterium extorquens CM4
atggccggcaacgagacgatcgaaacattcctcgacggcctggcgagctcggccccgacccccggcggcggcggtgccgccgcgatctccggcgccatgggcgcggcgctggtgtcgatggtgtgcaacctcaccatcggcaagaagaagtatgtcgaggtcgaggccgacctgaagcaggtgctggagaagtcggaaggcctgcgccgcacgctcaccggcatgatcgccgacgacgtcgaggctttcgacgcggtgatgggcgcctacgggctgccgaagaacaccgacgaagagaaggccgcccgcgccgccaagattcaagaggcgctcaaaaccgcgaccgacgtgccgctcgcctgctgccgcgtctgccgcgaggtgatcgacctggccgagatcgtcgccgagaagggcaatctcaacgtcatctcggatgccggcgtcgccgtgctctcggcctatgccggcctgcgctcggcggcccttaacgtctacgtcaacgccaagggcctcgacgaccgcgccttcgccgaggagcggctgaaggagctggagggcctactggccgaggcgggcgcgctcaacgagcggatctacgagaccgtgaagtccaaggtaaactga

SEQ ID NO: 3 - DNA, 867 bp, Methylobacterium extorquens
  source 1..867: mol_type = genomic DNA; note = Methylobacterium extorquens CM4
atgtccaagaagctgctcttccagttcgacaccgatgccacgccgagcgtcttcgacgtcgtcgtcggctacgacggcggtgccgaccacatcaccggctacggcaacgtcacgcccgacaacgtcggcgcctatgtcgacggcacgatctacacccgcggcggcaaggagaagcagtcgacggcgatcttcgtcggcggcggcgacatggcggccggcgagcgggtgttcgaggcggtgaagaagcgcttcttcggcccgttccgcgtgtcctgcatgctggattcgaacggctccaacacgaccgctgcggcgggcgtggcgctcgtcgtcaaggcggcgggcggctcggtcaagggcaagaaggccgtcgtgctcgcgggcaccggcccggtcggcatgcgctcggcggcgctgcttgccggcgagggcgccgaggtcgtgctgtgcgggcgcaagctcgacaaggcgcaggccgcggccgattccgtgaacaagcgcttcaaggtgaacgtcaccgcggccgagaccgcggacgacgcttcgcgtgccgaggccgtgaagggcgcccatttcgtcttcaccgccggtgcgatcggccttgaactgctgccgcaggcagcctggcagaacgagagttcgatcgagatcgtggccgactacaacgcccagccgccgctcggcatcggcgggatcgatgcgaccgacaaaggcaaggaatacggcggaaagcgcgccttcggtgcgctcggcatcggcggcttgaagctcaagctgcaccgcgcctgcatcgccaagctgttcgagtcgagcgaaggcgtcttcgacgccgaggagatctacaagctggccaaggaaatggcctga

SEQ ID NO: 4 - DNA, 1095 bp, Candida boidinii
  source 1..1095: mol_type = genomic DNA
atgaagatcgttttagtcttatatgatgctggtaagcacgctgctgatgaagaaaaattatatggttgtactgaaaataaattaggtattgctaattggttaaaagatcaaggtcatgaactaattactacttctgataaagaaggtgaaacaagtgaattggataaacatatcccagatgctgatattatcatcaccactcctttccatcctgcttatatcactaaggaaagacttgacaaggctaagaacttaaaattagtcgttgtcgctggtgttggttctgatcacattgatttagattatattaatcaaacaggtaagaaaatctcagtcttggaagttacaggttctaatgttgtctctgttgctgaacacgttgtcatgaccatgcttgtcttggttagaaatttcgttccagcacatgaacaaattattaaccacgattgggaggttgctgctatcgctaaggatgcttacgatatcgaaggtaaaactattgctaccattggtgctggtagaattggttacagagtcttggaaagattactcccttttaatccaaaagaattattatactacgattatcaagctttaccaaaagaagctgaagaaaaagttggtgctagaagagttgaaaatattgaagaattagttgctcaagctgatatcgttacagttaatgctccattacacgcaggtacaaaaggtttaattaataaggaattattatctaaatttaaaaaaggtgcttggttagtcaataccgcaagaggtgctatttgtgttgctgaagatgttgcagcagctttagaatctggtcaattaagaggttacggtggtgatgtttggttcccacaaccagctccaaaggatcacccatggagagatatgagaaataaatatggtgctggtaatgccatgactcctcactactctggtactactttagatgctcaaacaagatacgctgaaggtactaaaaatatcttggaatcattctttactggtaaatttgattacagaccacaagatattatcttattaaatggtgaatacgttactaaagcttacggtaaacacgataagaaataa

SEQ ID NO: 5 - DNA, 1155 bp, Arabidopsis thaliana
  source 1..1155: mol_type = genomic DNA
atggcgatgagacaagccgctaaggcaacgatcagggcctgttcttcctcttcttcttcgggttacttcgctcgacgtcagtttaatgcatcttctggtgatagcaaaaagattgtaggagttttctacaaggccaacgaatacgctaccaagaaccctaacttccttggctgcgtcgagaatgccttaggaatccgtgactggcttgaatcccaaggacatcagtacatcgtcactgatgacaaggaaggccctgattgcgaacttgagaaacatatcccggatcttcacgtcctaatctccactcccttccacccggcgtatgtaactgctgaaagaatcaagaaagccaaaaacttgaagcttctcctcacagctggtattggctcggatcatattgatctccaggcagctgcagctgctggcctgacggttgctgaagtcacgggaagcaacgtggtctcagtggcagaagatgagctcatgagaatcttaatcctcatgcgcaacttcgtaccagggtacaaccaggtcgtcaaaggcgagtggaacgtcgcgggcattgcgtacagagcttatgatcttgaagggaagacgataggaaccgtgggagctggaagaatcggaaagcttttgctgcagcggttgaaaccattcgggtgtaacttgttgtaccatcaaaggcatcagatggcaccagagctggagaaagagactggagctaagttcgttgaggatctgaatgaaatgctccctaaatgtgacgttatagtcatcaacatgcctctcacggagaagacaagaggaatgttcaacaaagagttgatagggaaattgaagaaaggcgttttgatagtgaacaacgcaagaggagccatcatggagaggcaagcagtggtggatgcggtggagagtggacacattggagggtacagcggagacgtttgggacccacagccagctcctaaggaccatccatggcgttacatgcctaaccaggctatgacccctcatacctccggcaccaccattgacgctcagctacggtatgcggcggggacgaaagacatgttggagagatacttcaagggagaagacttccctactgagaattacatcgtcaaggacggtgaacttgctcctcagtaccggtaa

SEQ ID NO: 6 - DNA, 987 bp, Streptococcus sp.
  source 1..987: mol_type = genomic DNA
atgaccgctactaagcagcacaagaaggtaattctggtaggtgacggtgcagttggttctagttacgcctttgcgctggttaatcaaggcatcgcgcaggaactgggtatcatcgagattccccagctttttgagaaagccgtcggggatgcgcttgacttgtcccatgcgttagctttcacctcaccgaaaaagatctacgcggcaaaatacgaggattgcgccgatgcagatttagtcgtaatcacagcaggggcgccgcagaagcccggcgagactcgtctggacctggtcgggaaaaatttggccattaacaaatcaatcgtcacccaagtcgttgaatcgggattcaacggtatatttcttgtagccgcaaaccccgttgacgtattaacttattcgacatggaaatttagtgggtttccaaaggaaagagtcataggttctggaacttctcttgatagcgcgagatttcgtcaagcactggctgagaagttaggcgttgatgctcgctccgttcatgcctacataatgggcgaacatggggactctgaatttgcagtttggtcccacgctaacatagctggtgtcaaccttgaagaattcctgaaagatacgcagaatgtccaggagagtgagttggtcgaattattcgagggagtgcgtgacgccgcttacacgattattaataaaaaaggggctacctattacggcattgcagttgcgcttgcccgtatcaccaaagcaatccttgacgatgagaatgcagttttgcctcttagcgtatttcaggaaggacaatacggagtgagtaacgtcttcataggacaaccggcaatcgtgggagcacacggaattgttcgtcccgttaatataccgcttaatgatgcggaacagcaaaagatgaaagcttcggctgatgagctgcaggccataatcgacgaagcctggaaaaacccagaattccaagaggcttccaagaattaa

SEQ ID NO: 7 - DNA, 51 bp, synthetic construct
  source 1..51: mol_type = other DNA
ttgctcctcagtaccggtaaaggaggaattcatgaccgctactaagcagca

SEQ ID NO: 8 - DNA, 44 bp, synthetic construct
  source 1..44: mol_type = other DNA
catccgccaaaacagccaagttaattcttggaagcctcttggaa

SEQ ID NO: 9 - DNA, 20 bp, synthetic construct
  source 1..20: mol_type = other DNA
ttgctcctcagtaccggtaa

SEQ ID NO: 10 - DNA, 23 bp, synthetic construct
  source 1..23: mol_type = other DNA
ttaattcttggaagcctcttgga